(19) United States
(12) Patent Application Publication
(10) Pub. No.: US 2019/0380563 A1
(43) Pub. Date: Dec. 19, 2019

(54) INTRODUCER SHEATH HAVING PERFUSION CAPABILITIES AND METHODS OF USE

(71) Applicant: Kintur Sanghvi, Princeton Junction, NJ (US)

(72) Inventor: Kintur Sanghvi, Princeton Junction, NJ (US)

(21) Appl. No.: 16/055,249

(22) Filed: Aug. 6, 2018

Related U.S. Application Data
(60) Provisional application No. 62/685,101, filed on Jun. 14, 2018.

(51) Int. Cl. A61B 1/00; A61M 25/00

(52) U.S. Cl. CPC A61B 1/00135; A61M 25/003; A61M 2025/0004; A61M 25/007

(57) ABSTRACT

An introducer sheath includes a flexible body having a proximal end and a distal end, the flexible body having a wall defining a central lumen extending from the proximal end to the distal end, a plurality of perfusion holes formed in the flexible body and spaced apart from one another, each of the plurality of perfusion holes being in communication with the central lumen of the flexible body, and a marker located on an outer surface of the wall closer to the proximal end of the flexible body than the plurality of perfusion holes.

### CROSS-REFERENCE TO RELATED APPLICATIONS

The present application claims priority to U.S. Provisional Application Ser. No. 62/685,101, entitled “PERFUSION INTRODUCER SHEATH TO ALLOW VASCULAR ACCESS FOR CARDIAC OR VASCULAR PROCEDURE, OR MECHANICAL CIRCULATORY SUPPORT WHILE ALLOWING BLOOD FLOW THORUGH THE INTRODUCER SHEATH IN THE ARTERY OR VEIN BEYOND THE POINT OF INTRODUCER SHEATH INSERTION,” filed Jun. 14, 2018, the contents of which are hereby incorporated by reference as if fully set forth herein.

The present disclosure relates to introducer sheaths for use during cardiac or vascular procedures. More specifically, the present disclosure relates to perfusion introducer sheaths, which allow vascular access for procedures, while permitting blood flow through the sheath in to the artery or vein beyond the point of introducer sheath insertion.

### BACKGROUND OF THE DISCLOSURE

Vascular introducer sheaths are typically used to access an artery or a vein during medical procedures. These procedures may include cardiac, endovascular, structural heart procedures, or mechanical circulatory support, amongst others. In many situations the outer diameter of the introducer sheath (often simply referred to as “introducer”) is larger or equal to the inner diameter of the artery or vein. As a result, blood flow in the artery distal is blocked at the point of introducer sheath insertion. Patients presenting with cardiovascular conditions or shock often have underlying peripheral artery disease, vasoconstriction, or small size arteries, which further increases the chances of the introducer outer diameter being larger than the inner diameter of the artery.

In some situations, large size introducer sheaths are left in the arterial system for long periods of time during which there is no blood flow beyond the point of sheath insertion. Impeding blood flow in this manner may lead to ischemia, cold limb, infection, metabolic disturbances, or gangrene of the body tissue perfused by that artery downstream. This may also lead to stagnant blood flow, higher risk of blood clot formation, embolization and/or arterial or venous occlusion. Often this is recognized only after the damage to the body tissue is done. Currently available introducer sheaths are flow-limiting. If physicians recognize the problem with such introducers early, it may require additional arterial access or an additional surgery to insert a cannula and create an external bypass circuit to perfuse the artery distal to the sheath.

Similarly, when small artery access is desired with small or regular-sized sheaths such as radial, ulnar, pedal, posterior tibial, the introducer sheath is flow limiting and thus may lead to stagnant blood, higher risk of thrombus formation, embolization and/or arterial occlusion. Thus, it would be beneficial to provide an introducer that does not impede blood flow beyond the insertion point.

### SUMMARY OF THE INVENTION

In some embodiments, an introducer sheath includes a flexible body having a proximal end and a distal end, the flexible body having a wall defining a central lumen extending from the proximal end to the distal end, a plurality of perfusion holes aligned with one another, formed in the flexible body and spaced apart from one another, each of the plurality of perfusion holes being in communication with the central lumen of the flexible body, and a marker located on an outer surface of the wall closer to the proximal end of the flexible body than the plurality of perfusion holes.

In some embodiments, an assembly includes an introducer sheath including a flexible body having a proximal end and a distal end, the flexible body having a wall defining a central lumen extending from the proximal end to the distal end, a plurality of perfusion holes aligned with one another, formed in the flexible body and spaced apart from one another, each of the plurality of perfusion holes being in communication with the central lumen of the flexible body, and a marker located on an outer surface of the wall closer to the proximal end of the flexible body than the plurality of perfusion holes, and a dilator sized for insertion through the introducer sheath.

In some embodiments, a method of introducing a tool into a patient's body includes piercing the patient's skin with a needle at an insertion point, advancing a wire through the needle into the body, removing the needle, advancing an introducer sheath including a flexible body having a proximal end and a distal end, the flexible body having a wall defining a central lumen extending from the proximal end to the distal end, a plurality of perfusion holes aligned with one another formed in the flexible body and spaced apart from one another, each of the plurality of perfusion holes being in communication with the central lumen of the flexible body, and a marker located on an outer surface of the wall closer to the proximal end of the flexible body than the plurality of perfusion holes, pulling the wire to remove it from the patient's body, and positioning the introducer sheath within the patient's body such that at least one of the perfusion holes is aligned with a central axis of an artery and blood is capable of passing through the at least one perfusion hole from the inside of the flexible body to the outside of the flexible body.

### BRIEF DESCRIPTION OF THE DRAWINGS

Various embodiments of the presently disclosed devices and methods are shown herein with reference to the drawings, wherein:

FIG. 1 is a schematic side view of an introducer sheath and a dilator according to one embodiment of the disclosure;

FIG. 2 is a schematic side view of a cannula and a dilator according to one embodiment of the disclosure;

FIG. 3 is a schematic side view of the dilator of FIG. 1 being inserted through the sheath of FIG. 1;

FIG. 4 is a schematic partial cross-sectional view of an introducer sheath according to one embodiment of the disclosure;

FIGS. 5 and 6 are schematic cross-sectional views of a cannula similar to that of FIG. 2 being used within an artery; and

FIGS. 7A-F are schematic representations showing the steps of using an introducer sheath.

It is to be appreciated that these drawings depict only some embodiments of the disclosure and are therefore not to be considered limiting of its scope.

### DETAILED DESCRIPTION OF THE DISCLOSURE

Despite the various improvements that have been made to introducer sheaths, conventional devices suffer from some shortcomings as described above.

The present disclosure describes introducers and cannulas that allow blood flow through them in to the artery or vein beyond the point of insertion and thus eliminate the risk of hypoperfusion, ischemia or gangrene. Such devices may eliminate the need of additional arterial access or surgery to create external bypass circuit. It will also allow continuous blood flow and reduce the risks of stagnation, clotting, embolism and occlusion.

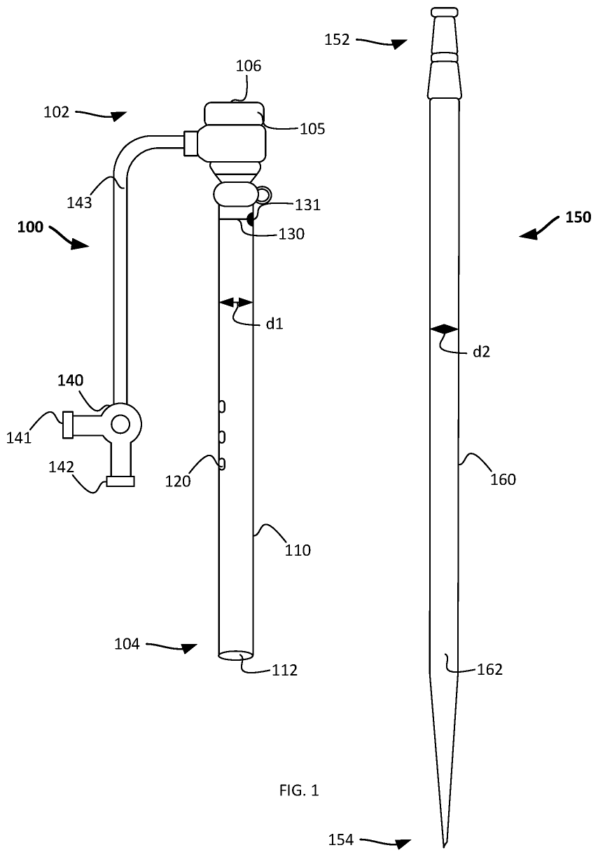

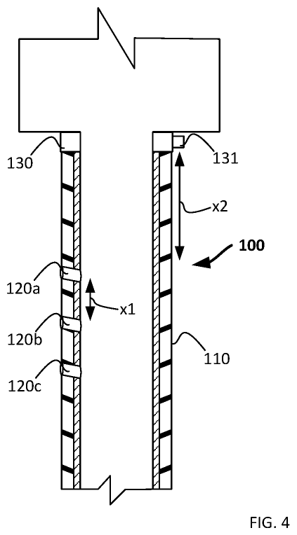

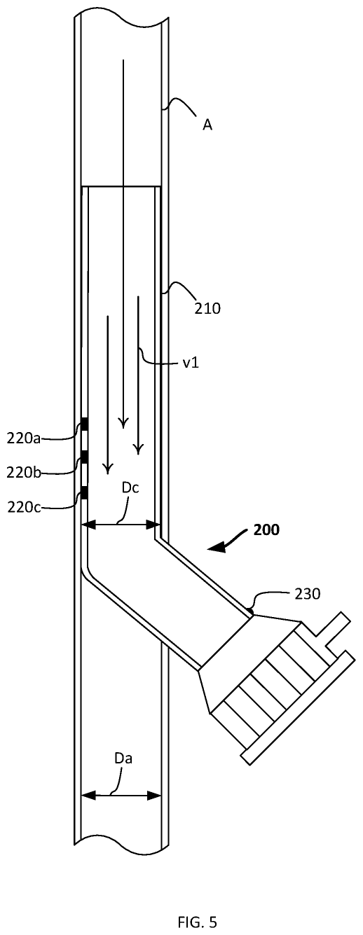

FIG. 1 is a schematic side view of an introducer sheath 100 and a dilator 150 for use with the introducer sheath. Sheath 100 has a proximal end 102 and a distal end 104 and includes a hub 205 having a hemostasis valve to limit blood and/or air transport, the hub being disposed adjacent proximal end 102, and having a central opening 106. A flexible body 110 extends from the hub to the distal end of the device, flexible body defining lumen 112 in communication with the central opening of the hub. Body 110 may be formed of a biocompatible polymer, plastic or other suitable material including nylon or similar compositions, layers of braided wire, PTFE, or others and may be flexible enough to bend within the vasculature. Lumen 112 may extend throughout the flexible body from one end of the sheath to the other and may be sized to accept an implement including dilator 150 and other tools, repair, rehabilitation or replacement devices that will be transported from outside a patient's body to the vasculature. Additionally, similar devices, methods and techniques may be used for mechanical circulatory support where blood is removed by a cannula in the vein, is transported through an external pump and injected back into an artery using a similar cannula. Body 110 may also include a plurality of perfusion holes 120 as shown in FIG. 1. In some examples, perfusion holes 120 include only a single hole. Alternatively, perfusion holes 120 include two or more holes, such as for example, three holes as illustrated. In some examples, three holes may be better for large bore sheaths, while two holes may be better for smaller sheaths. In a TAVR procedure, for example, three holes may be used to cover variation in the size of the femoral artery distribution in the population. Perfusion holes 120 may be defined in the sidewall of flexible body 110 and in communication with lumen 112 such that a liquid (e.g., blood) is capable of passing from the lumen to the outside of the flexible body. The distance “x1” between perfusion holes may be chosen as will be discussed in greater detail with respect to FIG. 4.

Sheath 100 may further include a marker 130 disposed closer to the proximal end 102 of the sheath than perfusion holes 120. In at least some examples, marker 130 may be disposed at or near a junction of the hub and the flexible body. In at least some embodiments, marker 130 is an annular band disposed about the flexible body 110. The marker 130 may have an annotation 131 disposed on a contralateral side of the flexible body with respect to the perfusion holes 120. That is, the annotation 131 may be spaced exactly 180 degrees from the axis on which the perfusion holes align, such that a physician that sees annotation 131 will understand that the perfusion holes are on the opposite side of the flexible body and ascertain their location without seeing them.

In at least some alternatives, marker 130 is not annular, but only includes a single annotation in the form of a, button, marking, symbol, indentation, or rib on the flexible body positioned contralateral to the perfusion holes so that the location of the perfusion holes is easily found.

The introducer sheath including the perfusion holes may be formed using known techniques such as injection molding/extrusion techniques. Alternatively, the perfusion holes may be formed using mechanical, lasers or other means after forming the introducer sheath. Alternatively, the proximal-most hole may have a radio-opaque marker. In some examples, the marker is also injection molded. Alternatively, a visible maker is created by using standard ink staining technique in the anterior wall at the junction of the tubular shaft and hemostatic valve.

As shown, sheath 100 further includes a secondary arm 140 having a pair of ports 141, 142 for receiving contrast, saline, medicaments and/or other substances, each of the ports being in communication with a tubing 143 that is in communication with lumen 112 of flexible body 110. Secondary arm 140 may optionally include a stopcock.

A dilator 150 is also shown in FIG. 1, the dilator having a proximal end 152, a distal end 154 and a body 160 extending between the two ends and defining a lumen 162. Lumen 162 may be sized to accept a guidewire therein. Dilator may have a diameter d2 that is slightly smaller than the diameter of flexible body 110 d1 so that the dilator is insertable within the sheath 100. As shown, body 160 of dilator 150 may have a taper adjacent the distal end to allow for smooth entry into the vasculature, the taper increasing from a relatively narrow diameter adjacent to the distal end to a large diameter d2 as shown.

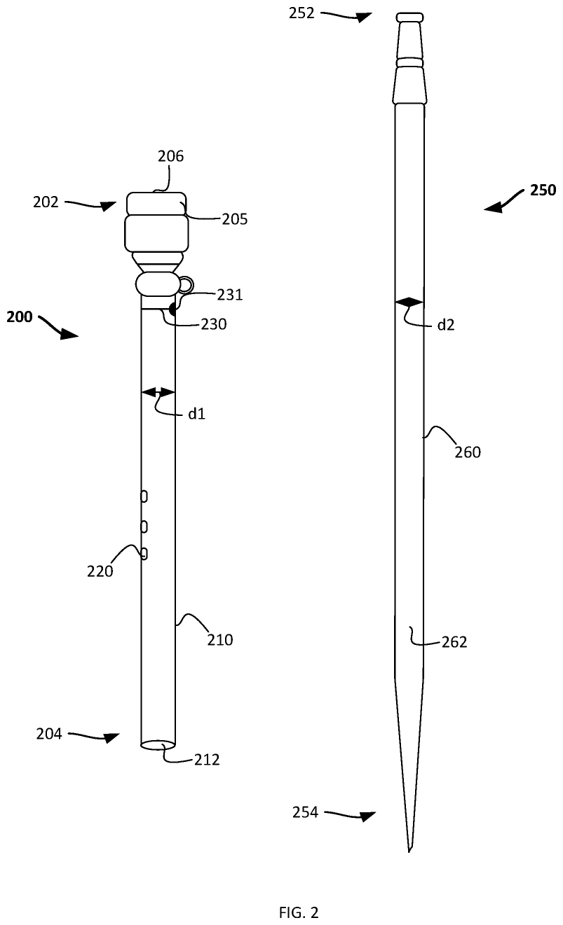

FIG. 2 is a schematic side view of a cannula 200 and a dilator 250 according to another embodiment of the disclosure. Like-numbered elements of FIG. 2 will be easily identified as being similar to those elements of FIG. 1, except that such elements will be preceded with “2” instead of “1”. It will be understood that while the previous embodiment shows an introducer sheath, the principles of the disclosure may be equally applicable to cannulas, tubings, catheters and other similar devices used within the vasculature. For example, perfusion holes 220 are shown on cannula 200 which are similar to perfusion holes 120 of sheath 100. It will be appreciated that cannula 200 is similar to sheath 100 in many ways (e.g., it includes a proximal and distal ends 202,204, a flexible body 210, a lumen 212, perfusion holes 220 and a marker 230), except that it does not include a side arm 140 or a number of ports. Likewise, dilator 250 for use with cannula 200 is similar to dilator 150 of FIG. 1.

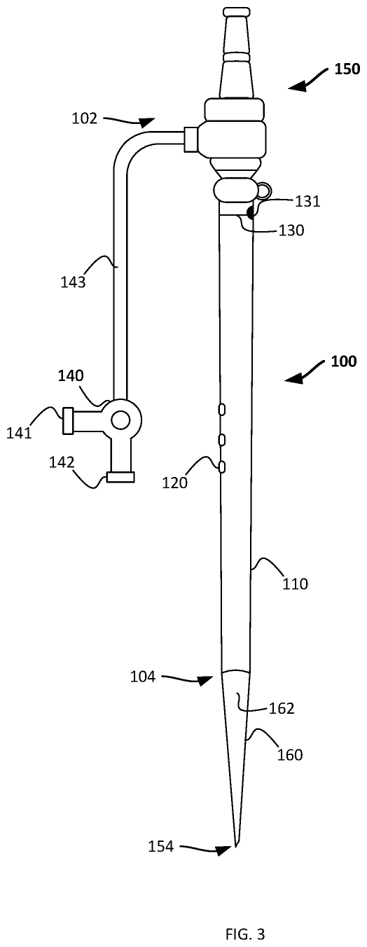

As discussed, the dilators are insertable within the sheath and/or cannula. FIG. 3 shows one example of a dilator 150 being disposed within lumen 112 of body 110 of the sheath 100. As shown, dilator 150 is slightly longer than sheath 100 such that the distal end of the dilator extends out of the sheath 100 when the dilator is inserted therein to its appropriate position. This along with the taper of the dilator may allow for easier insertion of the dilator-introducer combination.

The spacing between perfusion holes 120 and the marker 130 will be better understood by examining the cross-section shown in FIG. 4. As shown, perfusion holes extend through the wall of the body to permit communication between lumen 112 and the outside of the body. Each perfusion hole 120a, 120b, 120c may be between 0.1 to 2 mm in diameter.

A first spacing “x1” may be set between perfusion holes 120a, 120b, 120c. In at least some examples, the spacing “x1” between perfusion holes 120a and 120b is the same as the spacing “x1” between perfusion holes 120b and 120c. Alternatively, the spacing between the perfusion holes may be different from one another. In some embodiments, the spacing “x1” between perfusion holes may be between 2 mm and 5 mm. It will be understood that the spacing “x1” can be changed depending on the purpose of the introducer sheath, the size of the sheath, procedure to be performed, the expected thickness of tissue at the target site, the position of the vasculature in relation to the target site, and the intended arteries to be accessed by particular introducers.

A second spacing “x2” is defined as the distance between the marker 130 and the perfusion hole 120 that is closest to the marker. In at least some examples, spacing “x2” is between 1 cm and 7 cm. In some examples, the marker 130 is placed at the proximal-most end of the body and thus the proximal-most perfusion hole 120 is approximately 1 cm to 7 cm from the end of the body. It will be understood that the spacing “x2” can likewise be varied depending on the procedure to be performed, the expected thickness of tissue at the target site, the position of the vasculature in relation to the target site, and the intended arteries to be accessed by particular introducers.

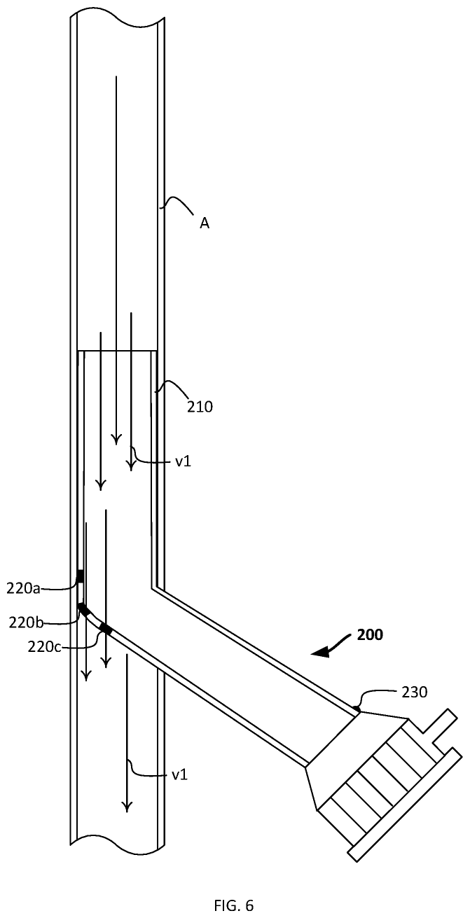

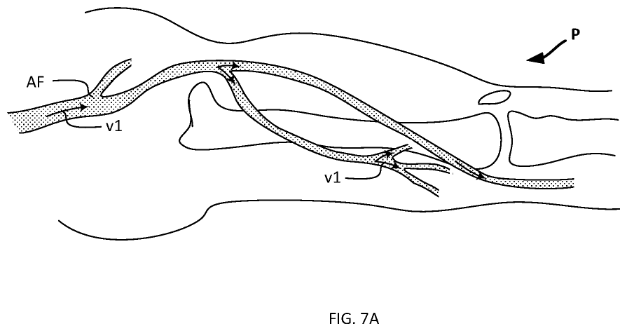

FIGS. 5 and 6 are schematic cross-sectional views of a cannula 100 similar to that of FIG. 2 being used within an artery “A”. Cannula 200 may have an outer diameter Dc that is approximately equal to the inner diameter of the artery Da. In this example, cannula 200 includes a flexible body 210 having a lumen 212, a plurality of perfusion holes 220a-c, and a marker 230 in the form of a raised button on the flexible body. As shown in FIGS. 5 and 6, in a first position (FIG. 5), cannula 200 is placed within artery “A” and perfusion holes 220a-c are disposed adjacent and against a wall of the artery “A”. In this position, antegrade blood flow v1 is capable of traveling through the artery and into the interior of cannula 100. Hemostasis valve within the hub of the cannula may prevent blood from flowing out of the cannula. Due to the cannula being disposed within the artery, antegrade blood flow does not continue past the cannula within the artery at positions downstream from the cannula.

Conversely, as shown in FIG. 6, when the cannula 200 is pulled back slightly, one or more of perfusion holes 220a-c travel from a position of being against the arterial wall and into the center of the artery. For example, perfusion holes 220c is now disposed near the center of the artery “A” and antegrade blood flow v1 may continue from the artery, through the lumen of the cannula 200, and out of the perfusion hole 220c back into the artery “A” at a position that is downstream from the cannula 200. In such a manner and when placed properly, cannula 200 may be positioned within the artery “A” to complete a procedure, and antegrade blood flow is permitted to points in the artery downstream of the cannula.

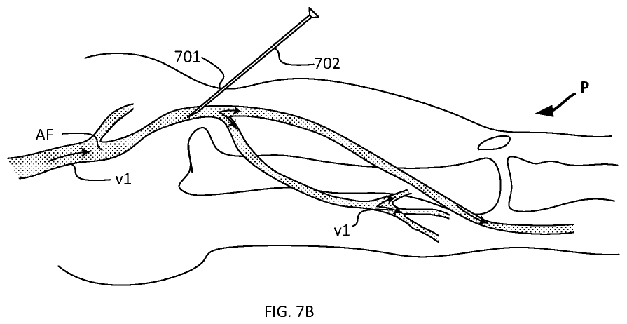

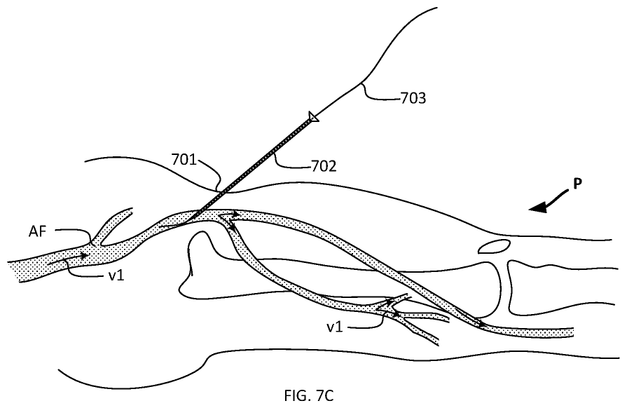

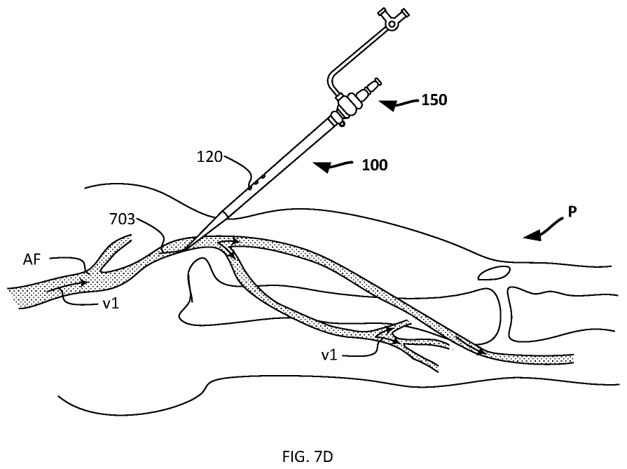

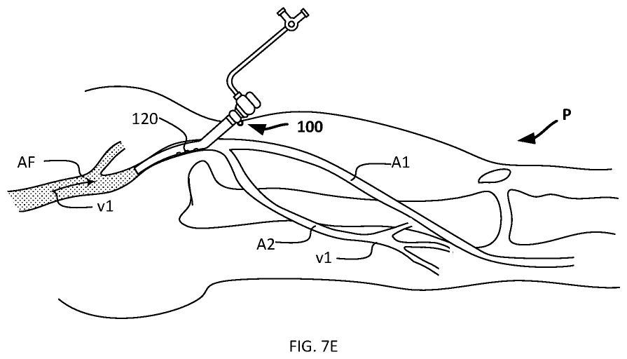

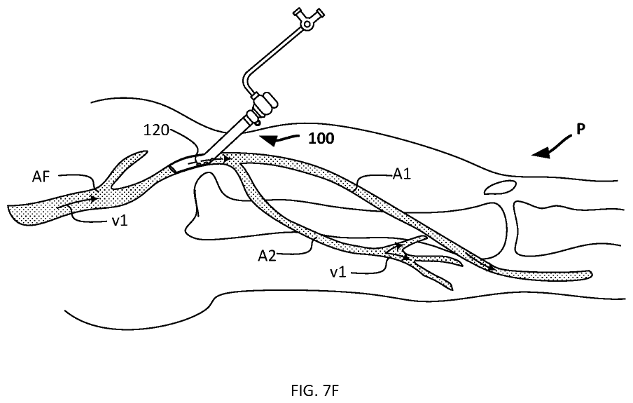

FIGS. 7A-F are schematic representations showing the steps of using an introducer sheath within patient “P” in a femoral artery “AF”. First, the location of the femoral artery “AF” is identified. In the initial condition, normal antegrade blood flow “v1” continues unimpeded (FIG. 7A). A hollow needle 702 is introduced at an insertion point 701 and guided into the femoral artery “AF” (FIG. 7B). A guidewire 704 is inserted through the center of hollow needle 702 and into the femoral artery (FIG. 7C). The needle 702 is removed while the guidewire 704 stays in place. In this instance, an introducer sheath 100 and a dilator 150 travel over guidewire 704 to a position at least partially within the femoral artery “AF” (FIG. 7D). The marker of the introducer sheath may be kept at the 12 o'clock position (e.g., away from the patient and visible to the physician) so that the positions of the perfusion holes are readily recognizable by the physician. When the introducer sheath is inserted in to the arterial system the proximal end of the body near the hemostatic valve remains outside the skin. Distal to that another 1 to 7 cm segment of the body remains in the subcutaneous tissue depending on the artery or vein accessed or patient body habitus. As the introducer sheath 100 advances, it beings to block blood flow into arteries that are downstream of the introducer sheath. The guidewire and the dilator may be removed from the patient, leaving the introducer sheath secured within the vasculature. For example, as seen in FIG. 7E, sheath 100 impedes blood from reaching downstream arteries A1 and A2.

Next, by gently withdrawing the introducer sheath 100 into a position in which perfusion holes 102a, 120b, and/or 120c are aligned within the center of the femoral artery AF, antegrade blood flow v1 may continue from the artery into the lumen of the sheath 100, through at least one of the perfusion holes and back out of the sheath to the downstream arteries A1, A2. This step may be performed with or without the aid of imaging systems.

For example, the port of the introducer sheath may be connected to a syringe full of contrast. While the contrast is injected the sheath is slowly withdrawn till the antegrade flow is seen in the artery beyond the point of insertion through the most proximal perforation. This is done carefully to avoid withdrawing the most proximal hole outside the artery. If the proximal-most perfusion hole does travel outside the artery there will be extravasation of contrast seen. In that case by using the dilator the sheath is inserted further into the artery and slow withdrawal is attempted again. In the cannulas or introducers without the side port, a syringe full of contrast may be connected with a small size dilator, or needle and contrast can be injected through the hemostatic valve for the same function. Similarly, the sheath may be pulled slowly back with ultrasound guidance to the point when the flow is seen beyond the point of insertion.

A similar technique may be used in venous systems so that blood will enter from these perforations holes and exit through the distal end in to the vein beyond the point of insertion. These same principles may be used for large-size introducer sheaths used for larger artery or vein access such as femoral, brachial, axillary, subclavian or carotid artery, as well as for the small or regular size introducer sheaths used for small artery access such as radial, ulnar, pedal, tibial, and other not mentioned here. Whenever the introducer outer diameter is larger or equal to the inner diameter of the vessel, it will allow the blood flow through the sheath in to the artery or vein and thus eliminate the risk of hypoperfusion tissue injury, stagnation of blood, thrombus formation, occlusion of the artery. Similar perfusion holes-marker arrangements may also be applied to delivery system for endo-prosthesis or in-line sheaths used for delivery of valves. Similar perfusion holes-marker arrangements may also be applied to endoprosthesis delivery sheaths, where perfusion holes could be applied at a distance dependent on the length of the endoprosthesis.

Although the invention herein has been described with reference to partcular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

It will be appreciated that the various dependent claims and the features set forth therein can be combined in different ways than presented in the initial claims. It will also be appreciated that the features described in connection with individual embodiments may be shared with others of the described embodiments.

What is claimed is:

1. An introducer sheath comprising:
a flexible body having a proximal end and a distal end, the flexible body having a wall defining a central lumen extending from the proximal end to the distal end;
a plurality of perfusion holes aligned with one another, formed in the flexible body and spaced apart from one another, each of the plurality of perfusion holes being in communication with the central lumen of the flexible body; and
a marker located on an outer surface of the wall closer to the proximal end of the flexible body than the plurality of perfusion holes.

2. The introducer sheath of claim 1, wherein the plurality of perfusion holes includes three perfusion holes.

3. The introducer sheath of claim 1, wherein each of the plurality of perfusion holes is between 0.1 mm and 2 mm in diameter.

4. The introducer sheath of claim 1, wherein each of the plurality of perfusion holes is spaced from an adjacent hole by between 2 mm and 5 mm.

5. The introducer sheath of claim 1, wherein the marker is spaced from a closest one of the plurality of perfusion holes by at least 1 cm to 7 cm.

6. The introducer sheath of claim 1, wherein at least one of perfusion holes has a radiopaque marker.

7. The introducer sheath of claim 1, wherein the marker includes an annular band disposed on the flexible body.

8. The introducer sheath of claim 1, wherein the flexible body is substantially cylindrical, and the plurality of perfusion holes are aligned together.

9. The introducer sheath of claim 1, wherein the plurality of perfusion holes is disposed at a first side of the flexible body, and the marker is at least partially disposed on a second side of the flexible body, the second side being contralateral to the first side.

10. An assembly comprising:
an introducer sheath including a flexible body having a proximal end and a distal end, the flexible body having a wall defining a central lumen extending from the proximal end to the distal end, a plurality of perfusion holes aligned with one another, formed in the flexible body and spaced apart from one another, each of the plurality of perfusion holes being in communication with the central lumen of the flexible body, and a marker located on an outer surface of the wall closer to the proximal end of the flexible body than the plurality of perfusion holes; and
a dilator sized for insertion through the introducer sheath.

11. The assembly of claim 10, wherein the plurality of perfusion holes includes three perfusion holes.

12. The assembly of claim 10, wherein the marker includes an annular band disposed on the flexible body.

13. The assembly of claim 10, wherein the flexible body is substantially cylindrical, and the plurality of perfusion holes are aligned together.

14. The assembly of claim 10, wherein the plurality of perfusion holes is disposed at a first side of the flexible body, and the marker is at least partially disposed on a second side of the flexible body, the second side being contralateral to the first side.

15. A method of introducing a tool into a patient's body comprising:
piercing the patient's skin with a needle at an insertion point;
advancing a wire through the needle into the body;
removing the needle;
advancing an introducer sheath including a flexible body having a proximal end and a distal end, the flexible body having a wall defining a central lumen extending from the proximal end to the distal end, a plurality of perfusion holes aligned with one another formed in the flexible body and spaced apart from one another, each of the plurality of perfusion holes being in communication with the central lumen of the flexible body, and a marker located on an outer surface of the wall closer to the proximal end of the flexible body than the plurality of perfusion holes;
pulling the wire to remove it from the patient's body; and
positioning the introducer sheath within the patient's body such that at least one of the perfusion holes is aligned with a central axis of an artery and blood is capable of passing through the at least one perfusion hole from the inside of the flexible body to the outside of the flexible body.

16. The method of claim 15, wherein positioning the introducer sheath within the patient's body includes introducing contrast into the sheath and slowly withdrawing the sheath until antegrade blood flow is seen in the artery beyond the point of insertion through the at least one perfusion hole.
